(19) United States
(12) Patent Application Publication
(10) Pub. No.: US 2005/0170478 A1
(43) Pub. Date: Aug. 4, 2005

(54) Expression of phospholipid:diacylglycerine acyltranssferase (pdat) for the production of plant storage lipids with polyunsaturated fatty acids

(76) Inventors: Sten Stymne, Svalov (SE); Marit Lenman, Lund (SE); Ulf Stahl, Uppsala (SE); Antoni Banas, Siedlce (PL); Anders Carlsson, Lund (SE); Eva Wiberg, Uppsala (SE); Anders Dahlqvist, Furulund (SE)

Correspondence Address: MORRISON & FOERSTER LLP, 1650 TYSONS BOULEVARD, SUITE 300, MCLEAN, VA, 22102, US

(21) Appl. No.: 10/509,796

(22) PCT Filed: Mar. 21, 2003
(86) PCT No.: PCT/EP03/02955

(30) Foreign Application Priority Data
Mar. 30, 2002 (DE) 102 14 410.9

(51) Int. Cl. C12P 7/64; C12N 9/10; A01H 1/00

(52) U.S. Cl. 435/134; 800/281; 435/193

(57) ABSTRACT

The present invention relates to the use of an enzyme mixture containing at least one enzyme with phospholipid:diacylglycerol acyltransferase activity for the production of plant storage lipids containing polyunsaturated fatty acids.

The present invention relates to the use of an enzyme mixture containing at least one enzyme with phospholipid:diacylglycerol acyltransferase activity for the production of plant storage lipids containing polyunsaturated fatty acids.

Triacylglycerol (TAG) constitutes the commonest fat-based energy store found in nature. Besides acyl-CoA:diacylglycerol acyltransferases (DAGAT), phospholipid:diacylglycerol acryltransferases (PDATs), which catalyze the synthesis of storage fats (triacylglycerol, TAG), are known to date (Dahlqvist et al., Proc. Natl. Acad. Sci., USA, 2000; 97: 6487-6492). The enzymes with PDAT activity catalyze, in an acyl-CoA-independent reaction, the transfer of acyl groups from the sn-2 position of the phospholipid to diacylglycerol (DAG), thus giving rise to TAG and a lysophospholipid.

Biochemical studies into this transfer reaction have already been carried out on seeds of Ricinus communis and Crepis palestina, both of which accumulate a high ricinoleic acid and vernolic acid content, respectively, and in sunflowers, whose seed oil contains only saturated fatty acids (Dahlqvist et al., Proc. Natl. Acad. Sci., USA, 2000; 97: 6487-6492).

In plants such as oilseed rape, sunflower, oil palm and the like, the oil (i.e. triacylglycerol) is the most valuable product of the seeds or fruit. Other constituents, such as starch, protein and fibers, are thought of as by-products of lesser value. Increasing the amount of oil on a weight basis, at the expense of other constituents, in oil plants would thus increase the value of the plant.

By modifying the activity of the genes which regulate the distribution of reduced carbon to oil production it would be feasible for the cells to accumulate more oil at the expense of other products. Such genes might be used not only in cells in which oil production is already high, such as, for example, oil plants, but might also induce substantial oil production in plants with a moderate or low oil content, such as, for example, soybeans, oats, maize, potatoes, sugarbeet or swedes, and in microorganisms.

Genes encoding a phospholipid:diacylglycerol acyl-transferase have previously been cloned from yeast (WO 00/60095). In yeast, PDAT shows dependency on the polar head groups of the donor lipid, the transferred acyl group, and the acyl chain of the acceptor molecule DAG. The increased expression of yeast genes encoding enzymes with PDAT activity in the homologous yeast system itself results in an increased oil content in the cells in question. In this context, it is mainly the monounsaturated fatty acids with hydroxyl, epoxy and acetylene groups which are removed from the membrane and converted into the storage lipid TAG (WO 00/60095).

WO 00/60095 furthermore also describes two nucleotide sequences of Arabidopsis thaliana. Transferring the Arabidopsis gene into yeast constituted functional proof for the fact that these Arabidopsis genes encode an enzyme with PDAT activity (WO 00/60095).

However, there is substantial interest worldwide not only in monounsaturated fatty acids, but also in polyunsaturated fatty acids (PUFAs) for large-scale use. These polyunsaturated fatty acids are of utmost economic interest for example for supplementing foods and feeds. Thus, a high content in lipids with unsaturated fatty acids, specifically polyunsaturated fatty acids, is important in the nutrition of animals and humans since the former additionally have a positive effect on the triglycerol level, or cholesterol level, and thus reduce the risk of heart disease. Unsaturated fatty acids are employed in a variety of dietetic foods or medicaments. Polyunsaturated fatty acids are essential nutrients since the human and animal organism is not capable of producing them itself. As a rule, polyunsaturated fatty acids are, however, not found in plants or if so then only in concentrations which are of no economic interest.

It is therefore an object of the present invention to provide polyunsaturated fatty acids from renewable plant resources, avoiding the disadvantages of traditional production methods such as, for example, production by the complicated fermentation of (microbial) single cells, distillation from fish oil or eco-unfriendly methods from non-renewable petrochemical products.

This object is achieved by the use of an enzyme mixture comprising at least one enzyme with phospholipid:diacylglycerol acyltransferase (PDAT) activity for the production of plant storage lipids containing polyunsaturated fatty acids.

It is furthermore possible, by the use according to the invention of an enzyme mixture containing at least one PDAT enzyme, together with at least one further enzyme for the synthesis of unusual fatty acids, to provide for example fatty acids with conjugated double bonds or long-chain polyunsaturated fatty acids.

“Polyunsaturated fatty acids” are understood as meaning those fatty acids with a chain length of at least 14 carbon atoms which have at least 3 double bonds. For the purposes of the present invention polyunsaturated fatty acids belong to the unusual fatty acids which are generally not found in plants.

The “unusual fatty acids” include, for example, fatty acids with hydroxyl, epoxy and acetylene groups, polyunsaturated fatty acids, preferably long-chain polyunsaturated fatty acids or fatty acids with conjugated double bonds. Those of interest are, for example, gamma-linolenic acid, arachidonic acid, stearidonic acid, eicosapentaenoic acid or docosahexaenoic acid, conjugated linolic acid or conjugated linolenic. acid (CLA). However, this enumeration is not limiting.

In the present invention, long-chain polyunsaturated fatty acids are preferred among the polyunsaturated fatty acids. “Long-chain polyunsaturated fatty acids are understood as meaning fatty acids with a chain length of at least 18 carbon atoms and at least 3 double bonds. Preferred fatty acids are those with at least 3 double bonds and with 18-24, especially preferably 18-22, in particular 20, carbon atoms. These include, for example, arachidonic acid, stearidonic acid, eicosapentaenoic acid, gamma-linolenic acid or docosahexaenoic acid. Arachidonic acid is preferred.

Conjugated fatty acids are understood as meaning fatty acids with at least 16 carbon atoms and at least 3 conjugated double bonds, such as, for example, CLA (conjugated linolenic acid).

A series of enzymes, such as, for example, hydroxylases, epoxygenases, acetylenases, desaturases, elongases, conjugases, trans-desaturases or isomerases, are involved in the synthesis of these unusual fatty acids. The resulting unusual fatty acids are incorporated into the membrane lipids by the plants.

Since unusual fatty acids do not normally occur in plant membranes, the maintenance of correct membrane function, and thus correct cellular function, must be ensured. Accordingly, the unusual fatty acids should only be present in low concentrations in the membrane lipids. This requires that the unusual fatty acids, once incorporated in the membrane lipids, are again removed therefrom efficiently. In the present invention, this is achieved by employing an enzyme mixture containing at least one enzyme with PDAT activity, the PDAT enzyme removing the unusual fatty acid from the membrane lipids and transporting it to the storage lipids (TAG) of the plant seeds.

The present invention furthermore encompasses the use of an enzyme mixture containing at least one enzyme with phospholipid:diacylglycerol acyltransferase activity and at least one further enzyme with the activity of a hydroxylase, epoxygenase, acetylenase, desaturase, elongase, conjugase, trans-desaturases or isomerases for producing plant storage lipids containing polyunsaturated fatty acids.

The coexpression of PDAT and further enzymes which are involved in the synthesis of unusual fatty acids brings about, in plants, a greater accumulation of unusual fatty acids in the storage lipids than is the case without PDAT.

In a preferred variant of the present invention, an enzyme mixture containing an enzyme with phospholipid:diacylglycerol acyltransferase activity and desaturase activity and elongase activity is used. This use can thus serve for the production of long-chain polyunsaturated fatty acids. Preferably, these are gamma-linolenic acid, arachidonic acid, eicosapentaenoic acid, stearidonic acid or docosahexaenoic acid.

Also encompassed within the invention is the use of an enzyme mixture containing an enzyme with phospholipid:diacylglycerol acyltransferase activity and desaturase activity or trans-desaturase activity for producing gamma-linolenic acid or conjugated linoleic acid. The use of an enzyme mixture containing at least one enzyme with phospholipid:diacylglycerol acyltransferase activity and one enzyme with conjugase, trans-desaturase or isomerase activity, is suitable in accordance with the invention for the production of conjugated fatty acids such as, for example, conjugated linolenic acid (CLA).

The synthesis, in plants, of polyunsaturated fatty acids, their incorporation into the membrane lipids and the conversion into storage lipids of the plant seeds are achieved by increasing at least the activity of one PDAT enzyme. This can be brought about by increased gene expression, increased catalytic enzyme activity or modified regulatory enzyme activity. The skilled worker is familiar with measures required for this purpose.

In accordance with the invention, the expression of a gene encoding a PDAT enzyme together with at least one further gene encoding an enzyme of the group of hydroxylases, epoxygenases, acetylenases, desaturases, elongases, conjugases, trans-desaturases or isomerases is required for the synthesis of unusual fatty acids, such as long-chain polyunsaturated or conjugated fatty acids.

Increased expression of a gene can be achieved by a procedure with which the skilled worker is familiar. These procedures include, for example, increasing the copy number of the gene in question by at least a factor of 2, advantageously by a factor of 5-10. The replicating nucleotide sequence may be chromosomally or extrachromosomally encoded according to the invention.

Operative linkage with regulatory sequences may furthermore be mentioned. They may influence transcription, RNA stability or RNA processing, and translation. Examples of regulatory sequences are, inter alia, promoters, enhancers, operators, terminators or translation enhancers. They may take the form of natural regulatory sequences or modified regulatory sequences. The amplification of regulatory sequences is also feasible.

Increased gene expression, in this context, is to be viewed with regard to an endogenously (naturally) present enzyme activity. Also encompassed for the purposes of the present invention is heterologous gene expression, that is to say the expression of one or more genes which do not naturally occur in plants, in which case increased gene expression is to be regarded as an increase over a value of zero.

A modified, preferably increased, catalytic and/or modified regulatory activity of PDAT enzymes or of further enzymes which are involved in the synthesis of unusual fatty acids can be undertakes by recombinant modifications of the coding sequence in question, or by what is known as molelcular modeling. The measures required for this purpose are standard laboratory practice for the skilled worker.

What has been said before regarding the increased expression/enzyme activity also applies to the enzyme(s) which is/are present in a plant cell in addition to phospholipid:diacylglycerol acyltransferase in order to increasingly convert unusual fatty acids into storage lipids.

In an advantageous embodiment of the present invention, a nucleotide sequence encoding a plant-derived enzyme with PDAT activity is used. The isolated nucleotide sequence encoding an enzyme with PDAT activity is preferably derived from Arabidopsis thaliana.

In a further variant of the present invention, the enzyme with PDAT activity encompasses an amino acid sequence as shown in SEQ ID No. 2 encoded by a nucleotide sequence as shown in SEQ ID No. 1, or alleles thereof.

These sequences are disclosed in WO 00/60095 as the sequence referred to as AB006704.

In accordance with the invention, an isolated nucleic acid, or an isolated nucleic acid fragment, is understood as meaning an RNA or DNA polymer which can be single- or double-stranded and may optionally contain natural, chemically synthesized, modified or artificial nucleotides. In this context, the term DNA polymer also includes genomic DNA, cDNA or mixtures of these.

In accordance with the invention, alleles are understood as meaning functionally equivalent nucleotide sequences i.e. nucleotide sequences with essentially the same action. Functionally equivalent sequences are those sequences which retain the desired function despite a deviating nucleotide sequence, for example owing to the degeneracy of the genetic code. Thus, functional equivalents encompass naturally occurring variants of the sequences described herein, but also artificial nucleotide sequences, for example nucleotide sequences which have. been obtained by chemical synthesis and which, if appropriate, have been adapted to suit the codon usage of the host organism. In addition, functionally equivalent sequences encompass those with a modified nucleotide sequence which imparts to the enzyme for example desensitivity or resistance to inhibitors.

A functional equivalent is also to be understood as meaning, in particular, natural or artificial mutations of a sequence which has originally been isolated, which mutations continue to show the desired function. Mutations encompass substitutions, additions, deletions, exchanges or insertions of one or more nucleotide residues. Also included are what are known as sense mutations, which, at the protein level, for example lead to the substitution of conserved amino acids, but which do not lead to a principal change in the activity of the protein and are thus neutral with regard to its function. They also include modifications of the nucleotide sequence which, at the protein level, concern the N- or C terminus of a protein, but without, however, having a major adverse effect on the function of the protein. Indeed, these modifications may have a stabilizing effect on protein structure.

Other nucleotide sequences which are also encompassed by the present invention are, for example, those which are obtained by modification of the nucleotide sequence, resulting in corresponding derivatives. The purpose of such a modification may be, for example, the further delimitation of the coding sequence contained therein, or else, for example, the insertion of further cleavage sites for restriction enzymes. Functional equivalents are also those variants whose function is weakened or increased in comparison with the starting gene, or gene fragment.

Also subject of the present invention are artificial DNA sequences, as long as they impart the desired characteristics, as described above. Such artificial DNA sequences can be identified for example by back-translation of proteins generated by means of computer-aided programs (molecular modeling), or by in vitro selection. Especially suitable are coding DNA sequences which have been obtained by backtranslating a polypeptide sequence in accordance with the host-organism-specific codon usage. The skilled worker who is familiar with molecular-genetic methods can readily determine the specific codon usage by means of computer evaluations of other, known genes of the organism to be transformed.

Homologous sequences are understood as meaning, in accordance with the invention, those which are complementary to the nucleotide sequences according to the invention and/or which hybridize therewith. In accordance with the invention, the term hybridizing sequences includes substantially similar nucleotide sequences from among the group of DNA or RNA, which enter a specific interaction (binding) with the. abovementioned nucleotide sequences under stringent conditions which are known per se. A preferred, nonlimiting example for stringent hybridization conditions is hybridization in 6× sodium chloride/sodium citrate (SSC) at approximately 45° C. followed by one or more wash steps in 0.2×SSC, 0.1% SDS at 50-65° C. Also included are short nucleotide sequences of, for example, 10 to 30 nucleotides, preferably 12 to 15 nucleotides. Primers or hybridization probes are likewise included.

A homologous nucleotide sequence for the purposes of the present invention is a sequence which has at least approximately 40%, preferably at least approximately 50% or 60%, particularly preferably at least approximately 70%, 80% or 90% and most preferably at least approximately 95%, 96%, 97%, 98% or 99% or more homology with a nucleotide sequence as shown in SEQ ID No. 1.

Also included in accordance with the invention are the sequence regions which proceed (5′, or upstream) the coding regions (structural genes) and/or which follow (3′, or downstream) the same. They include, in particular, sequence regions which have a regulatory function. They can affect transcription, RNA stability, RNA processing or else translation. Examples of regulatory sequences are, inter alia, promoters, enhancers, operators, terminators or translation enhancers.

The present invention furthermore relates to a gene structure containing at least one nucleotide sequence encoding a phospholipid:diacylglycerol acryltransferase and regulatory sequences which are linked operably therewith and which govern the expression of the coding sequences in the host cell.

Examples of suitable host cells are plant cells or algal cells, or microorganisms such as E. coli, yeast or filamentous fungi.

Operable linkage is understood as meaning the sequential arrangement of, for example, promoter, coding sequence, terminator and, if appropriate, further regulatory elements in such a way that each of the regulatory elements can fulfill its intended function upon expression of the coding sequence. These regulatory nucleotide sequences can be of natural origin or else be obtained by chemical synthesis. A suitable promoter is, in principle, any promoter capable of governing gene expression in the host organism in question.

The following may be mentioned by way of example: the cauliflower mosaic virus promoter CaMV35S (Frank et al., 1980, Cell, 21: 285) or the B. napus napin promoter (Stalberg et al., 1993, Plant Molecular Biology, 23:671-683). In accordance with the invention, this promoter may also be a chemically inducible promoter by means of which the expression of the genes which it controls can be controlled, in the host cell, at a particular point in time. Other advantageous promoters are those which permit tissue-specific expression, preferably seed-specific expression. Examples which may be mentioned are the following promoters: the USP promoter (Bäumlein et al., 1991, Mol. Gen. Genet., 225 (3): 459-467, the oleosin promoter (WO 98/45461) or the B4 promoter from legumes (LeB4; Bäumlein et al., 1992, Plant Journal, 2 (2): 233-239.

A gene structure is generated by fusing a suitable promoter to at least one nucleotide sequence according to the invention, using customary recombination and cloning techniques as are described, for example, in T. Maniatis, E. F. Fritsch and J. Sambrook, Molecular Cloning: A Laboratory Manual, Cold Spring Harbor Laboratory, Cold Spring Harbor, NY (1989) or Kaiser et al., 1994, Methods in Yeast Genetics, Cold Spring Harbor Laboratory Press or Guthrie et al., 1994, Guide to Yeast Genetics and Molecular Biology, Methods in Enzymology, Academic Press.

Adaptors or linkers may be attached to the fragments to connect the DNA fragments with one another.

In addition, the present invention relates to a vector containing at least one nucleotide sequence of the above-described type encoding a phospholipid:diacylglycerol acyltransferase, regulatory nucleotide sequences linked operably to the former, and additional nucleotide sequences for the selection of transformed host cells, for replication within the host cell or for integration into the relevant host cell genome. Moreover, the vector according to the invention may contain a gene structure of the abovementioned type.

Vectors which are suitable are, for example, those which are replicated in microorganisms or plants. The following enumeration is not limiting for the present invention: PGEX (Pharmacia Biotech, Inc.; Smith et al., 1988, Gene 67:31-40), pMAL (New England Biolabs, Beverly, Mass.), pRIT5 (Pharmacia, Piscataway, N.J.) with glutathione S-transferase (GTS), maltose binding protein or proteinA, pTrc(Amann et al., 1988, Gene 69: 301-315), pET vectors (Sudier et al., Genen Expression Technology, Methods in Enzymology 185, Academic Press, San Diego, Calif., 1990: 60-89 and Strategene, Amsterdam, Netherlands), pYepSec1 (Baldari et al., 1987, Embo J. 6: 229-234), pMFa (Kurjan et al., 1982, Cell 30: 933-943), pJRY88 (Schultz et al., 1987, Gene 54: 113-123), pYES derivatives (Invitrogen Corporation, San Diego, CA) or vectors for use in filamentous fungi are described in: van den Hondel, C. A. M. J. J. & Punt, Pl. (1991) “Gene transfer systems and vector development for filamentous fungi, in: Applied Molecular Genetics of Fungi, J. F. Peberdy et al., eds., p. 1-28, Cambridge University, Press: Cambridge. Examples of plant expression vectors are also found in Becker, D., et al. (1992) “New plant binary vectors with selectable markers located proximal to the left border, Plant Mol Biol 20: 1195-1197 or in Bevan, M W. (1984) “Binary Agrobacterium vectors for plant transformation”, Nucl. Acid. Res. 12: 8711-8721. As an alternative, insect cell expression vectors may also be used, for example for expression in Sf 9 cells. They are, for example, the vectors of the pAc series (Smith et al. (1983) Mol Cell Biol 3:2156-2165) and vectors of the pVL series (Lucklow and Summers (1989) Virology 170:31-39). Further expression vectors are pCDM8 and pMT2PC, which are mentioned in: Seed, B. (1987) Nature 329:840 or Kaufman et al., (1987) EMBO J. 31 6: 187-195. Promoters preferably to be used are of viral origin, such as, for example, promoters of polyoma virus, adenovirus 2, cytomegalovirus or simian virus 40. Further prokaryotic and eukaryotic expression systems are mentioned in Chapters 16 and 17 in Sambrook et al., Molecular Cloning: A Laboratory Manual 2^{nd}, ed., Cold Spring Harbor Laboratory, Cold Spring Harbor Laboratory Press, Cold Spring Harbor, N.Y., 1989.

Using the nucleic acid sequences according to the invention, it is also possible to synthesize suitable probes or else primers which can be used for amplifying and isolating analogous genes from other organisms, for example with the aid of PCR technology.

In addition, the present invention relates to the amino acids with PDAT activity which are derived from the nucleic acids. These also include isoenzymes of PDAT. Isoenzymes are understood as meaning enzymes which have the same or a similar substrate specificity and/or catalytic activity, but a different primary structure. Also encompassed in accordance with the invention. are modified forms of PDAT. In accordance with the invention, these are understood as meaning enzymes in which modifications are present in the sequence, for example at the N and/or C terminus of the polypeptide or in the region of conserved amino acids, but without adversely affecting the function of the enzyme. These modifications can be carried out by methods known per se in the form of amino acid substitution.

A particular embodiment of the present invention also encompasses the use of variants of the PDAT according to the invention whose activity is weakened or enhanced in comparison with the original protein in question, for example owing to amino acid substitution. The same applies to the stability of the enzyme according to the invention in cells which are more or less susceptible to, for example, degradation by proteases.

Also subject of the present invention are polypeptides with the function of a PDAT whose amino acid sequence is modified in such a way that they are desensitive to regulatory compounds, for example to catabolites which regulate their activity (feedback desensitive).

The present invention furthermore also includes the transfer of a nucleic acid sequence as shown in SEQ ID No. 1 or part thereof which encodes a PDAT or an allele, homolog or derivative thereof into a host system. The transfer of a gene construct or vector according to the invention into a suitable host system is also included. The transfer of foreign genes into the genome of the plant is referred to as transformation. Generally customary methods for the transformation and regeneration of plants from plant tissues or plant cells are exploited for transient or stable transformation.

Suitable methods are protoplast transformation by polyethylene glycol-induced DNA uptake, the biolistic method using the gene gun (what is known as the particle bombardment method), electroporation, incubation of dry embryos in DNA-containing solution, microinjection and the agrobacterium-mediate gene transfer. The abovementioned methods are described, for example, in B. Jenes et al., Techniques for Gene Transfer, in: Transgenic Plants, Vol. 1, Engineering and Utilization, edited by S. D. Kung and R. Wu, Academic Press (1993) 128-143, in Potrykus Annu. Rev. Plant Physiol. Plant Molec. Biol. 42, (1991), 205-225, Moloney et al., 1992, Plant Cell Reports, 8: 238-242, Mlynarova et al., 1994, Plant Cell Report, 13: 282-285 and Bell et al., 1999, In Vitro Cell. Dev. Biol.-Plant., 35 (6): 456-465.

The construct to be expressed is preferably cloned into a vector which is suitable for transforming Agrobacterium tumefaciens, for example pBinI9 (Bevan et al., Nucl. Acids Res. 12 (1984) 8711). Agrobacteria transformed with such a vector can then be used in a known manner for transforming plants, in particular crop plants, such as, for example, tobacco plants, for example by bathing scarified leaves or leaf sections in an agrobacterial solution and subsequently growing them in suitable media. The transformation of plants with Agrobacterium tumefaciens is described, for example, by Höfgen and Willmitzer in Nucl. Acid Res. (1988), 16, 9877 or is known from, inter alia, F. F. White, Vectors for Gene Transfer in Higher Plants; in Transgenic Plants, Vol. 1, Engineering and Utilization, edited by S. D. Kung and R. Wu, Academic Press, 1993, 5, 15-38.

Agrobacteria transformed with a vector according to the invention can likewise be used in the manner known per se for transforming plants such as laboratory plants such as Arabidopsis or crop plants such as cereals, maize, oats, rye, barley, wheat, soybean, rice, cotton, sugar beet, canola, sunflower, flax, hemp, potato, tobacco, tomato, carrot, capsicum, oilseed rape, tapioca, cassava, arrowroot, tagetes, alfalfa, lettuce and the various tree, nut and vine species, in particular oil-containing crop plants such as soybean, peanut, castor-oil plant, sunflower, maize, cotton, flax, oilseed rape, coconut, oil palm, safflower (Carthamus tinctorius) or cocoa bean, for example by bathing scarified leaves or leaf segments in an agrobacterial solution and subsequently growing them in suitable media.

The genetically modified plant cells can be regenerated by all methods which are known to the skilled worker. Suitable methods can be found in the abovementioned publications by S. D. Kung and R. Wu, Potrykus or Höfgen and Willmitzer.

The present invention also encompasses host organisms into which at least one of the abovementioned nucleotide sequences encoding a phospholipid:diacylglycerol acyltransferase and/or a corresponding gene construct and/or a corresponding vector of the abovementioned type has been transferred. In addition, the host organisms may additionally also contain nucleotide sequences which encode enzymes which participate in the synthesis of unusual fatty acids. Again, these nucleotide sequences may be of natural origin or generated synthetically. Moreover, they can be genetically modified and present in a comparable gene construct and/or vector of the abovementioned type. In this context, it is feasible that the nucleotide sequences encoding a phospholipid:diacylglycerol acyltransferase and enzymes for the synthesis of unusual fatty acids are present in one gene construct and/or vector or else in different gene constructs or vectors. In these transgenic host organisms according to the invention, in which the production of unusual fatty acids is enhanced in comparison with a corresponding, untransformed host organism, the nucleotide sequence encoding a phospholipid:diacylglycerol acyltransferase is present in an increased quantity, at least 2 copies and/or is expressed to a higher degree starting from upstream regulatory sequences. Furthermore, the phospholipid:diacylglycerol acyltransferase activity in a host organism which has been transformed in accordance with the invention may be increased as compared to the untransformed wildtype, which is firstly due to an increased amount of enzymes which are present in the cell or else, inter alia, due to a phospholipid:diacylglycerol acyltransferase whose catalytic activity has been modified. The regulation of the enzyme activity may furthermore also be modified.

In accordance with the invention, the host organisms, in principle, take the form of all organisms which are capable of synthesizing fatty acids and in the present context specifically unusual fatty acids, such as polyunsaturated, longer-chain unsaturated or conjugated fatty acids, or of organisms which are suitable for the expression of recombinant genes. They are preferably plants or plant cells, preferably useful plants or their cells. Plants which are preferred in accordance with the invention are Arabidopsis, Asteraceae such as Calendula, or crop plants such as soybean, peanut, castor-oil plant, sunflower, maize, cotton, flax, linseed, thistles, oilseed rape, coconut, oil palm, safflower (Carthamus tinctorius) or cocoa bean. However, microorganisms such as fungi, for example the genus Mortierella, Saprolegnia or Pythium, bacteria such as the genus Escherichia, yeasts such as the genus Saccharomyces, cyanobacteria, ciliates, algae or protozoans such as dinoflagellates or Crypthecodinium, are also feasible.

Organisms which are preferred are those which are naturally capable of synthesizing oils in substantial amounts, for example fungi such as Mortierella alpina, Pythium insidiosum or plants such as soybean, oilseed rape, coconut, oil palm, safflower, castor-oil plant, Calendula, peanut, cocoa bean, sunflower, or yeasts such as Saccharomyces cerevisiae.

Utilizable host cells are furthermore mentioned in: Goeddel, Gene Expression Technology: Methods in Enzymology 185, Academic Press, San Diego, Calif. (1990).

The host organisms are grown or cultured in a manner with which the skilled worker is familiar. As a rule, microorganisms are grown in a liquid medium which contains a carbon source, usually in the form of sugars, a nitrogen source, usually in the form of organic nitrogen sources such as yeast extract or salts such as ammonium sulfate, trace elements such as iron salts, manganese salts, or magnesium salts and, if appropriate, vitamins at temperatures of between 0° C. and 100° C., preferably between 10° C. and 60° C., while passing in oxygen gas. In this context, the pH value of the liquid medium can be kept constant, that is to say regulated during the culture period, or not. Culturing can be carried out batchwise, semibatchwise or continuously. Nutrients can be introduced at the beginning of the fermentation or else fed semicontinuously or continuously.

To generate transgenic plants, binary vectors in Agrobacterium tumefaciens or Escherichia coli are made use of, for example. For the transformation, a 1:50 dilution of an overnight culture of a transformed agrobacteria colony in Murashige-Skoog medium (MS medium; Murashige and Skoog, 1962, Physiol. Plant, 15: 473) supplemented with 3% of sucrose (3MS medium) is made use of. Petioles or hypocotyls of freshly germinated sterile plants (in each case approx. 1 cm²) are incubated for 5-10 minutes in a 1:50 agrobacteria dilution in a Petri dish. This is followed by coincubation in the dark for 3 days at 25° C. on 3MS medium supplemented with Bacto agar. After 3 days, culturing was continued at 16 hours light/8 darkness, and continued in a weekly rhythm on MS medium supplemented with Claforan (cefotaxime-sodium), antibiotic, benzylaminopurine (BAP) and glucose. The young shoots are transferred to MS medium supplemented with 2% sucrose (2MS medium), Claforan and Batch agar. If no roots develop, the growth hormone 2-indolebutyric acid is added to the medium to induce rooting. Regenerated shoots are obtained on 2MS medium supplemented with antibiotic and Claforan; after the shoots have rooted, they are transferred into soil and, after having been grown for 2 weeks, grown in a controlled-environment cabinet or in the greenhouse, allowed to flower, and mature seeds are harvested and their fatty acid content is analyzed.

In accordance with the invention, the storage lipids (triacylglycerols, TAGs) of the transgenic organisms according to the invention show an increased content of unusual fatty acids, such as polyunsaturated fatty acids, long-chain polyunsaturated fatty acids or conjugated fatty acids. In this context, the content of these fatty acids is increased in comparison with the fatty acid content which is normally found in the storage lipids of these plants.

After the transgenic organisms according to the invention have been grown, the lipids are obtained in the customary manner. To this end, the organisms can first be disrupted after they have been harvested or else used directly. The lipids are advantageously extracted using suitable solvents such as apolar solvents such as hexane or ethanol, isopropanol or mixtures such as hexane/isopropanol, phenol/chloroform/isoamyl alcohol, at temperatures of between 0° C. and 80° C., preferably between 20° C. and 50° C. As a rule, the biomass is extracted with an excess of solvent, for example a 1:4 excess of solvent to biomass. The solvent is subsequently removed, for example via distillation. Alternatively, the extraction may be carried out with supercritical CO₂. After the extraction, the remainder of the biomass can be removed for example by filtration.

The crude oil thus obtained can subsequently be purified further, for example by removing cloudiness by treatment with polar solvents such as acetone or chloroform, followed by filtration or centrifugation. Further purification through columns is also possible. To obtain the free fatty acids from triglycerides, the latter are hydrolzyed in the customary manner.

The invention thus also relates to unusual polyunsaturated, longer-chain polyunsaturated or conjugated fatty acids and to triglycerides with an increased content of these fatty acids which have been produced by the abovementioned procedures, and to their use for the production of foods, animal feeds, cosmetics or pharmaceuticals. To this end, the former are added to the foods, the animal feeds, the cosmetics or the pharmaceuticals in customary amounts.

In one variant of the present invention Arabidopsis thaliana plants with the nucleotide sequence as shown in SEQ ID No. 1 (AB006704) were transformed. These plants are subsequently analyzed for their novel characteristics:

Northern blot analyses

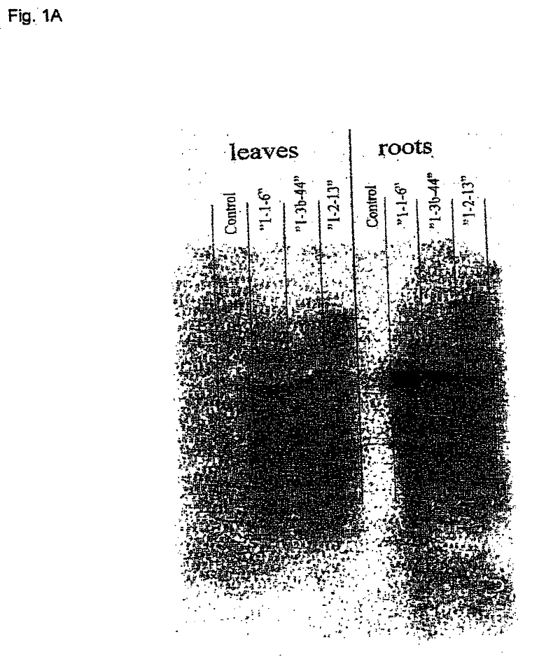

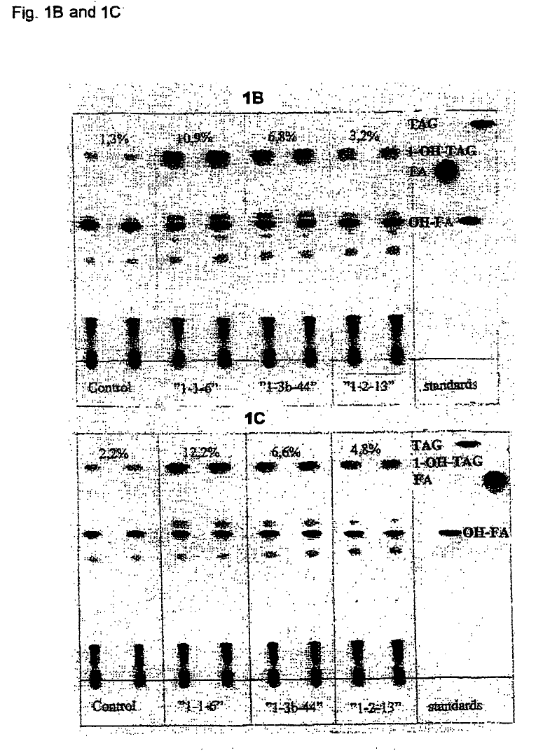

T2 plants which had been transformed with a control vector or with a vector containing the gene AB006704 were employed for the RNA extraction. Since the T2 seedlings revealed segregation with regard to the inserted gene, kanamycin was employed to eliminate untransformed seedlings. T2 seedlings of A. thaliana C24 transformed with the control vector, and T2 seedlings of 3 different 35S-AB006704 A. thaliana cv. Columbia transformants which had survived after germinating on kanamycin plates, were grown in liquid culture and used for the RNA extraction. RNA was prepared from leaves and roots and separated in a Northern blot (FIG. 1a). The expression of the Arabidopsis AB006704 gene the leaves and roots of A. thaliana C24 (transformed with the control vector) was barely detectable. The expression of the AB006704 gene was clearly visible in all of the 3 35S-AB006704 transformants, not only in the leaves but also in the roots, the highest expression level being observed in roots. AB006704 was expressed at the highest expression rate in transformant no. 1-1-6, the transformants 1-3b-44 and 1-2-13 showing bands with approximately 70% and 25%, respectively, of the intensity of the hybridization band of transformant 1-1-6.

PDAT enzyme assay

PDAT activity was determined in microsomal preparations of leaves and roots of T2 plants of A. thaliana C24 (transformed with the control vector) and of T2 plants of 3 different 35S-AB006704 transformants of A. thaliana cv. Columbia. Plants used for the preparation of microsomes were grown under the same conditions as plants which were used for the detection of AB006704 MRNA by Northern blot analyses. In earlier experiments (Dahlqvist et al., 2000, Proc. Natl. Acas. Sci., USA, 97:6487-6492; data not shown), PC with ricinoleic acid in position sn-2 was, in most cases, one of the best substrates for PDAT-catalyzed reactions.

The content of de novo-synthesized 1-OH-TAG generally represents the expression pattern of the AB006704 gene in plant material which was used for microsomal preparation (FIGS. 1b and 1c). Microsomes of transformant 1-1-6 (with the highest level of AB006704 gene expression) produced more TAG then microsomes of transformant 1-3b-44 (medium expression level). The microsomes of transformant 1-3b-44 synthesized more TAG than microsomes of transformant 1-2-13 (low expression level). Moreover, the microsomes of transformant 1-2-13 produced more TAG than microsomes which had been transformed with the blank control vector for control purposes.

AB006704 encodes an enzyme with PDAT activity

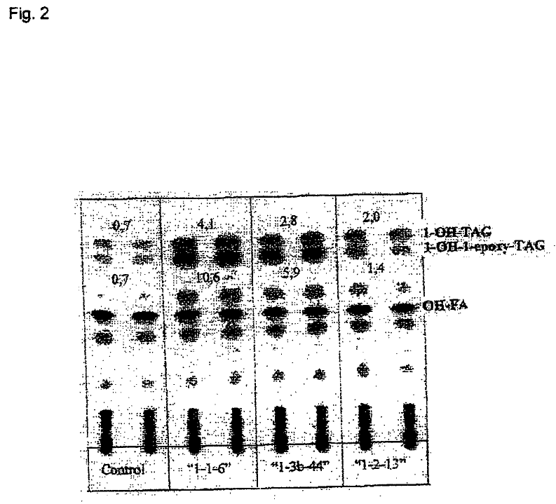

To demonstrate that the formation of ¹⁴C-labeled TAG from the previous experiment takes place via a PDAT-catalyzed reaction with PC as fatty acid donor and DAG as acyl acceptor, sn-1-oleoyl-sn-2-epoxy-DAG was employed as acyl acceptor and sn-1-oleoyl-sn-[¹⁴C]ricinoleoyl-PC as acyl donor in this experiment. Enzyme assays were carried out with the same microsomal preparation of leaves of T2 plants of A. thaliana C24 (transformed with the control vector) and T2 plants of 3 different transformants of A. thaliana cv. Columbia (with the AB006704 gene under the control of the 35S promoter) as was the case in the previous experiment. The de novo synthesis of TAG molecules containing both of the [¹⁴C]-ricinoleoyl and -vernoloyl groups (FIG. 2) clearly demonstrate the expression pattern of the AB006704 gene in plant material which had been used for the preparation of microsomes (FIG. 1a). The data obtained clearly demonstrates that the transgenic PDAT gene is capable of exploiting fatty acids from the sn-2 position of PC to acyl-sn-1-oleoyl-sn-2-epoxy-DAG for the formation of TAG. Here, the hydroxy- and epoxy-fatty acids in the transgenic plants containing the gene AB006704 are incorporated better into triacylglycerides (TAGs) than in the control plants. Accordingly, it has been demonstrated that the gene AB006704 encodes an enzyme with PDAT activity and is capable of exploiting PC as intermediate acyl donor and DAG as acyl acceptor in an acyl-CoA-independent reaction for the formation of TAG.

Substrate specificity

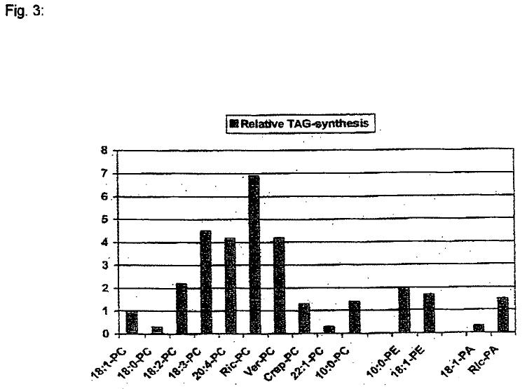

To study the substrate specificity (FIG. 3) of the A. thaliana protein encoded by the AB006704 gene, a microsomal preparation from leaves of transformant 1-1-6, which had been grown in liquid culture, was used for the assay. Protein AB006704 shows a higher activity toward PC with unsaturated fatty acids in position sn-2 than toward PC with saturated fatty acids. Among the 18-C fatty acids, PC with linolenic acid (18:3) in position sn-2 was the best substrate, while stearic acid (18:0) was transferred least. Moreover, for example, erucic acid (22:1) was transferred much less than oleic acid (18:1); arachidonic acid (20:4) was transferred at approximately the same rate as linolenic acid (18:3), even though arachidonic acid has one more double bond than linolenic acid.

Ricinoleic acid, which contains a hydroxyl group at position 12, was transferred to DAG with the highest efficacy of all acyl groups tested. Also, vernolic acid which contained an epoxy group, was transferred from the sn-2 position of PC to DAG with approximately twice the efficiency of the corresponding linoleic acid. Moreover, the A. thaliana PDAT which was studied showed differences in specificity toward phospholipids with head groups of different polarity. Phosphatidylethanolamine (PE) was utilized somewhat better than phosphatidylcholine (PC). The transfer of 10:0, or 18:1, by PE was approximately 1.5 times more rapid than in the case of PC. In contrast, phosphatidylic acid (PA) was a worse substrate than PC. The transfer of oleic and ricinoleic acid from position sn-2 of PA was 3 to 5 times less efficient than in the case of PC. Acyl compounds of the acceptor have the same effect on the activity of the A. thaliana PDAT which has been studied. For example, sn-1-oleoyl-sn-2-epoxy-DAG is a somewhat better acceptor than dioleoyl-DAG (FIG. 1B and FIG. 2).

This means that not only the chain length but also the number of double bonds and likewise the functional group in the vicinity of the head group has an effect on enzyme activity.

The present invention is illustrated hereinbelow by further examples which, however, do not limit the invention:

1. General methods

The isolation of plasmid DNA from bacteria or plants and all techniques regarding restriction, treatment with Klenow and alkaline phosphatase, electrophoresis, transformation, sequencing, RNA analyses or PCR were carried out as described by Sambrook et al. (Molecular cloning. A laboratory manual (1989) Cold Spring Harbour Laboratory Press).

2. Preparation of gene constructs and vectors

The nucleotide sequence AB006704 (SEQ ID No. 1), which encodes a PDAT enzyme from Arabidopsis thaliana (SEQ ID No. 2), was placed under the control of the 35S promoter.

Using restriction digestion, a NotI fragment was isolated from the ligation product and cloned into the binary vector pART27 (Lee et al., 1998, Science, 280:915-918) downstream of the napin promoter (for seed-specific expression). The vector pART27 without the nucleotide sequence encoding A. thaliana PDAT acted as a control vector.

3. Transformation of Arabidopsis thaliana

The above-described vector pART27 containing AB006704 was transferred into A. thaliana cv. Columbia plants by means of vacuum infiltration (Bent et al., 2000, Plant Physiol. 124, (4): 1540-1547). A corresponding control vector was transformed into an A. thaliana cv. C24. T1 seeds were seeded on ⅓ MS plates containing 1% sucrose and 50 μg/ml kanamycin. Grown seedlings were transplanted into soil, and T2 seeds were harvested.

To verify whether the gene AB006704 encodes the PDAT enzyme, constructs for direct expression of the AB006704 gene in plants were generated. To this end, the gene was cloned into a binary vector pART27, either downstream of the CaMV 35S promoter or the B. napus napin promoter, and Arabidopsis thaliana (cv. Columbia) was transformed by means of vacuum infiltration. T2 seedlings were analyzed for PDAT activity, and the expression of the gene AB006704 was studied with the aid of Northern blot analyses.

4. Microsomal preparation

T2 seeds of A. thaliana cv. C24 transformed with the blank vector and T2 seeds of A. thaliana cv. Columbia transformed with a vector containing AB006704 under the control of the 35S promoter were seeded on ⅓ MS plates containing 1% sucrose and 50 μg/ml kanamycin. Untransformed A. thaliana seeds (cv. Columbia) were seeded on plates without kanamycin. After 10 days, the seedlings which had grown were transferred into culture vessels containing liquid containing ½ MS containing 1% sucrose and incubated for 27 days at 23° C. in the light on a shaker. Microsomes of leaves and roots of the A. thaliana seedlings which had grown in liquid culture were prepared using the method of Stobart and Stymne (Biochemical Journal, 1985, 232 (1): 217-221).

5. Preparation of lipid substrates

Radiolabeled ricinoleic acid (12-hydroxy-9-octadecenoic acid) and vernolic acid (12,13-epoxy-9-octadecenoic acid) were synthesized enzymatically from [1-¹⁴C]oleic acid and [1-¹⁴C]-linoleic acid, respectively, by incubation with microsomal preparations from Ricinus communis or Crepis palaestina seeds, respectively (Bafor et al., 1991, Biochem. J. 280:507-514). Radiolabeled crepenynic acid (9-octadecen-12-ynoic acid) were synthesized enzymatically from [-1-¹⁴C]-linolic acid by incubation with microsomal preparations from Crepis alpina (Lee et al., 1998, Science, 280: 915-918). Radiolabeled 10:0, 18:0, 18:1, 18:2, 18:3, 20:1 and 20:4 fatty acids are commercially available. The synthesis of phosphatidylcholines (PC), phosphatidylethanolamines (PE) and phosphatidylic acid (PA) with ¹⁴C-labeled acyl groups in position sn-2 was carried out either by enzymatic acylation (Banas et al., 1992, Plant Science, 84: 137-144) or synthetic acylation. Sn-1-oleoyl-sn2-epoxy-DAG was prepared from Crepis palaestina triacylglycerols by treatment with lipase and separation via thin-layer chromatography.

6. Enzyme assay

Aliquots of crude microsomal fractions (corresponding to 12 nmol microsomal PC) from developing plant seeds, were lyophilized overnight. ¹⁴C-labeled substrate lipids (2.5 nmol of PC, PE or PA with ¹⁴C-labeled acyl groups in position sn-2 and 1.5 nmol of dioleoyl- or sn1-oleoyl-sn2-epoxy-DAG) which had been dissolved in benzene were then added to dried microsomes. The benzene was evaporated by passing in N₂, whereby the lipids were brought into direct contact with the membranes, and 0.1 ml of 50 mM potassium phosphate (pH 7.2) was added. The suspension was mixed thoroughly and incubated at 30° C. over the period stated of up to 60 min. The lipids were extracted from the reaction mixture using chloroform and separated. by thin-layer chromatography using silica gel 60 plates (Merck) in hexane/diethyl ether/ acetic acid (35:70:1.5 v/v) using silica gel 60 plates (Merck). The radioactive lipids were visualized on the plates via autoradiography (Instant Imager, Packard, USA) and quantified.

7. Growth experiments

Untransformed A. thaliana seedlings (cv. Columbia) and T2 seedlings of A. thaliana (cv. Columbia) transformed with the vector containing AB006704 under the control of the 35S promoter (plant 1-1-6 with the gene AB006704 with the highest expression) were seeded on 4 different ⅓ MS plates containing 1% sucrose (one half of the plates were seeded with control seedlings, while the other half was seeded with transformed seedlings) and grown for 17 days at 23° C. with exposure to light. The fresh weight of each seedling (approximately 50 seedlings of each type) was determined. Controls and transformed seedlings for each plate were collected and employed for lipid analysis.

8. Fatty acid content and lipid analysis

The fatty acid in A. thaliana wild-type (cv. Columbia) seedlings and in corresponding transformants was determined by extracting the lipids following the method of Bligh & Dyer (1959, Can. J. Biochem. Physiol., 37, 911-917) followed by methylation with 2% H₂SO₄ in dried methanol (60 minutes at 90° C.). Lipids in the Arabidopsis seedlings (2-3 mg/sample) were methylated directly with 2 ml of 2% strength (v/v) H₂SO₄ in dried methanol (90 minutes at 90° C.). The methyl esters were extracted with hexane and analyzed via GLC using “Chrompack” capillary columns (WCOT fused-silica column 50 m×0.32 mm ID coated with CD wax 58-CB DF=0.2). To quantify the fatty acid content, methylheptadecanoic acid was used as the internal standard.

Key to figures:

The present invention will additionally be illustrated hereinbelow with reference to the figures:

FIG. 1A shows an RNA (Northern blot) analysis of total RNA from leaves and roots of A. thaliana C24 control plants transformed with the blank control vector and three different A. thaliana plants transformed with the vector containing a nucleotide sequence as shown in SEQ ID No. 1 (PDAT) under the control of the 35S promoter.

FIG. 1B and FIG. 1C show the conversion of sn-1-oleoyl-sn-2-[¹⁴C]-ricinoleyl-PC during incubation for 1 hour with microsomes (and unlabeled sn-1-oleoyl-sn-2-oleoyl-DAG) from leaves (FIG. 1B) and roots (FIG. 1C) of A. thaliana C24 control plants and three different A. thaliana plants transformed with the vector containing a nucleotide sequence as shown in SEQ ID No. 1 (PDAT) under the control of the 35S promoter. The average radioactivity present in the de novo-synthesized 1-OH-TAG is shown in the autoradiograph as a percentage.

FIG. 2 shows the in vitro synthesis of TAG containing a vernoloyl and a [¹⁴C]-recinoleoyl group in microsomes of leaves of A. thaliana C24 control plant and three different A. thaliana plants transformed with the vector containing a nucleotide sequence as shown in SEQ ID No. 1 (PDAT) under the control of the 35S promoter. The substrates added are sn-1-oleoyl-sn-2-[¹⁴ ]-epoxy-DAG and sn-1-oleoyl-sn-2-[¹⁴C]-ricinoleoyl-PC. The average radioactivity present in the de novo-synthesized 1-OH-TAG and 1-OH-1-epoxy-TAG is shown in the autoradiographs as a percentage.

FIG. 3 shows the substrate specificity of the protein with PDAT activity which is encoded by the nucleotide sequence as shown in SEQ ID No. 1. Microsomal preparations of leaves of the A. thaliana transformant 1-1-6 transformed with the vector containing a nucleotide sequence as shown in SEQ ID No. 1 (PDAT) under the control of the 35S promoter were employed. Dioleoyl-DAG together with sn-1-oleoyl-sn-2-[¹⁴C]-fatty acid phospholipids (PC, PE, PA) were employed as substrate (18:0-PC, 20:4-PC, 22:1-PC, 10:0-PC and 10:0-PE with 16:0 in position sn-1). Relative enzyme activities of PADAT towards various substrates are shown, the activity towards 18:1-PC being designated 1 (20:4 is arachidonic acid).

Also appended is a sequence listing containing SEQ ID No. 1 and SEQ ID No. 2 for a PDAT enzyme from Arabidopsis thaliana.

1. A method for production of plant storage lipids containing polyunsaturated fatty acids comprising providing an enzyme mixture containing at least one enzyme with phospholipid:diacylglycerol acyltransferase activity.

2. The method of claim 1, wherein the enzyme mixture further contains at least one further activity of a hydroxylase, epoxygenase, acetylenase, desaturase, elongase, conjugase, trans-desaturase, isomerase or combination thereof.

3. The method of claim 1, wherein the enzyme mixture further contains desaturase activity and elongase activity.

4. The method of claim 1, wherein the polyunsaturated fatty acids comprise long-chain polyunsaturated fatty acids.

5. The method of claim 1, wherein the polyunsaturated fatty acids comprise one or more of gamma-linolenic acid, arachidonic acid, gamma-limolenic acid, eicosapentaenoic acid, stearidonic acid or docosahexaenoic acid.

6. The method of claim 1, wherein the at least one enzyme is encoded by a nucleotide sequence which is capable of replication, is present in a plant cell in at least 2 copies or contains regulatory sequences that bring about an at least 2-fold increase in gene expression or enzyme activity.

7. The method of claim 6, wherein the nucleotide sequence is encoded chromosomally or extrachromosomally.

8. The method of claim 6, wherein the nucleotide sequence is derived from plants.

9. The method of claim 6, wherein the nucleotide sequence is derived from Arabidopsis thaliana.

10. The method of claim 1, wherein the at least one enzyme comprises the amino acid sequence of SEQ ID No. 2.

11. The method of claim 1, wherein the at least one enzyme or a part thereof is encoded by the nucleotide sequence of SEQ ID No. 1 or alleles thereof.

12. The method of claim 1, wherein the polyunsaturated fatty acids contain fatty acids with conjugated double bonds.

13. The method of claim 1, wherein the polyunsaturated fatty acids comprise fatty acids with a chain length of at least 14 carbon atoms and having at least 3 double bonds.

14. The method of claim 1, wherein the polyunsaturated fatty acids comprise fatty acids not naturally found in plants.

15. A method for producing polyunsaturated acids from a plant comprising:
increasing a phospholipid:diacylglycerol acyltransferase activity of said plant; and
isolating the polyunsaturated fatty acids.

16. The method of claim 15, wherein increasing comprises increasing the copy number of a gene that encodes a phospholipid:diacylglycerol acyltransferase enzyme.

17. The method of claim 15, wherein increasing comprises increasing the catalytic or regulatory activity of one or more enzymes involved in synthesis of fatty acids.

18. The method of claim 15, wherein increasing comprising transforming said plant with a nucleotide sequence.

19. The method of claim 18, wherein the nucleotide sequence comprises SEQ ID No. 1 or a homolog or allele thereof.

20. The method of claim 19, wherein the homolog has a sequence which is at least 60% identical to said nucleotide sequence.


SEQUENCE LISTING (2 sequences)

SEQ ID NO: 1 - DNA, 2425 bp, Physcomitrella patens
  CDS (120)..(2135): PhospholipidDiacylglycerin-Acyltransferase
agaaacagctctttgtctctctcgactgatctaacaatccctaatctgtgttctaaattcctggacgagatttgacaaagtccgtatagcttaacctggtttaatttcaagtgacagatatgccccttattcatcggaaaaagccgacggagaaaccatcgacgccgccatctgaagaggtggtgcacgatgaggattcgcaaaagaaaccacacgaatcttccaaatcccaccataagaaatcgaacggaggagggaagtggtcgtgcatcgattcttgttgttggttcattgggtgtgtgtgtgtaacctggtggtttcttctcttcctttacaacgcaatgcctgcgagcttccctcagtatgtaacggagcgaatcacgggtcctttgcctgacccgcccggtgttaagctcaaaaaagaaggtcttaaggcgaaacatcctgttgtcttcattcctgggattgtcaccggtgggctcgagctttgggaaggcaaacaatgcgctgatggtttatttagaaaacgtttgtggggtggaacttttggtgaagtctacaaaaggcctctatgttgggtggaacacatgtcacttgacaatgaaactgggttggatccagctggtattagagttcgagctgtatcaggactcgtggctgctgactactttgctcctggctactttgtctgggcagtgctgattgctaaccttgcacatattggatatgaagagaaaaatatgtacatggctgcatatgactggcggctttcgtttcagaacacagaggtacgtgatcagactcttagccgtatgaaaagtaatatagagttgatggtttctaccaacggtggaaaaaaagcagttatagttccgcattccatgggggtcttgtattttctacattttatgaagtgggttgaggcaccagctcctctgggtggcgggggtgggccagattggtgtgcaaagtatattaaggcggtgatgaacattggtggaccatttcttggtgttccaaaagctgttgcagggcttttctctgctgaagcaaaggatgttgcagttgccagagcgattgccccaggattcttagacaccgatatatttagacttcagaccttgcagcatgtaatgagaatgacacgcacatgggactcaacaatgtctatgttaccgaagggaggtgacacgatatggggcgggcttgattggtcaccggagaaaggccacacctgttgtgggaaaaagcaaaagaacaacgaaacttgtggtgaagcaggtgaaaacggagtttccaagaaaagtcctgttaactatggaaggatgatatcttttgggaaagaagtagcagaggctgcgccatctgagattaataatattgattttcgaggtgctgtcaaaggtcagagtatcccaaatcacacctgtcgtgacgtgtggacagagtaccatgacatgggaattgctgggatcaaagctatcgctgagtataaggtctacactgctggtgaagctatagatctactacattatgttgctcctaagatgatggcgcgtggtgccgctcatttctcttatggaattgctgatgatttggatgacaccaagtatcaagatcccaaatactggtcaaatccgttagagacaaaattaccgaatgctcctgagatggaaatctactcattatacggagtggggataccaacggaacgagcatacgtatacaagcttaaccagtctcccgacagttgcatcccctttcagatattcacttctgctcacgaggaggacgaagatagctgtctgaaagcaggagtttacaatgtggatggggatgaaacagtacccgtcctaagtgccgggtacatgtgtgcaaaagcgtggcgtggcaagacaagattcaacccttccggaatcaagacttatataagagaatacaatcactctccgccggctaacctgttggaagggcgcgggacgcagagtggtgcccatgttgatatcatgggaaactttgctttgatcgaagatatcatgagggttgccgccggaggtaacgggtctgatataggacatgaccaggtccactctggcatatttgaatggtcggagcgtattgacctgaagctgtgaatatcatgatctctttaagctgtcctgtcagcttatgtgaatccaatactttgaaagagagatcatcatcaattcatcatcatcgtcatcatcatgatgctcaactcacaaagaagcctgagaatgatactttggtgcgaaattctcaatacctctttaatattcttattgaatgtaaattatacaatcctatctaatgtttgaacgataacacaaaacttgctgcgccatgtttgtttgtcttgtcaaaagcatcaatttgtgggttaaaaaaaaaaaaaaaaaaaaaa

SEQ ID NO: 2 - protein, 671 aa, Physcomitrella patens
MPLIHRKKPTEKPSTPPSEEVVHDEDSQKKPHESSKSHHKKSNGGGKWSCIDSCCWFIGCVCVTWWFLLFLYNAMPASFPQYVTERITGPLPDPPGVKLKKEGLKAKHPVVFIPGIVTGGLELWEGKQCADGLFRKRLWGGTFGEVYKRPLCWVEHMSLDNETGLDPAGIRVRAVSGLVAADYFAPGYFVWAVLIANLAHIGYEEKNMYMAAYDWRLSFQNTEVRDQTLSRMKSNIELMVSTNGGKKAVIVPHSMGVLYFLHFMKWVEAPAPLGGGGGPDWCAKYIKAVMNIGGPFLGVPKAVAGLFSAEAKDVAVARAIAPGFLDTDIFRLQTLQHVMRMTRTWDSTMSMLPKGGDTIWGGLDWSPEKGHTCCGKKQKNNETCGEAGENGVSKKSPVNYGRMISFGKEVAEAAPSEINNIDFRGAVKGQSIPNHTCRDVWTEYHDMGIAGIKAIAEYKVYTAGEAIDLLHYVAPKMMARGAAHFSYGIADDLDDTKYQDPKYWSNPLETKLPNAPEMEIYSLYGVGIPTERAYVYKLNQSPDSCIPFQIFTSAHEEDEDSCLKAGVYNVDGDETVPVLSAGYMCAKAWRGKTRFNPSGIKTYIREYNHSPPANLLEGRGTQSGAHVDIMGNFALIEDIMRVAAGGNGSDIGHDQVHSGIFEWSERIDLKL